(19) United States
(12) Patent Application Publication
(10) Pub. No.: US 2020/0143266 A1
(43) Pub. Date: May 7, 2020

(54) ADVERSARIAL BALANCING FOR CAUSAL INFERENCE

(71) Applicant: International Business Machines Corporation, Armonk, NY (US)

(72) Inventors: Tal El-Hay, Modi'in (IL); Michal Ozery-Flato, Tel-Aviv (IL); Pierre Thodoroff, Villennes Sur Seine (FR)

(21) Appl. No.: 16/182,635

(22) Filed: Nov. 7, 2018

(51) Int. Cl. G06N 5/02; G06N 99/00; G06N 7/00; G16H 50/20

(52) U.S. Cl. CPC G06N 5/027; G16H 50/20; G06N 7/005; G06N 99/005

(57) ABSTRACT

Embodiments of the present systems and methods may provide techniques for measuring similarity between two datasets using classification error as a measure of the similarity between the two datasets and for improving the similarity between the two datasets. For example, in an embodiment, a computer-implemented method for determining treatment effects may comprise receiving data relating to observations of treatments outcomes of at least one treatment in a plurality of treatment groups, wherein the data for each treatment group forms a dataset, reweighting at least some of the datasets to balance biases in the data among the datasets by: determining bias between at least two datasets using a classification error; and generating balancing weights for at least one of the datasets to reduce the bias between the at least two dataset, and determining treatment effects using at least one reweighted dataset.

### BACKGROUND

The present invention relates to techniques for measuring similarity between two datasets and reweighting at least one dataset to increase the similarity between the two datasets. In particular, classification error may be used as a measure of the similarity between two datasets.

Biases in observational data pose a major challenge to treatment effect estimation methods. An important technique that accounts for these biases is reweighting samples to minimize the discrepancy between treatment groups. The inverse propensity weighting, which models the conditional treatment probability given covariates, is a common weighting technique, but has been shown to be sensitive to model miss-specification. Recent methods attempt to find weights that minimize a discrepancy measure between the reweighted populations.

Accordingly, a need arises for techniques that may provide improved solutions for generating weights for a given dataset to maximize the similarity of the weighted dataset to a given target dataset.

### SUMMARY

Embodiments of the present systems and methods may provide techniques for measuring similarity between two datasets using classification error as a measure of the similarity between the two datasets and for improving the similarity between the two datasets. Embodiments may utilize bi-level optimization of minimizing classification error on one hand, and on the other hand generating weights that maximize classification error and hamper the ability to distinguish between the two datasets. In embodiments, a model may be trained to discriminate between the treatment groups and use its classification error as a measure of similarity between them. The key idea is that the larger the classification error is, the more indistinguishable treatment groups are. Embodiments may include a new framework for generating balancing weights that uses a bi-level optimization to alternately: (i) train a discriminator to minimize classification error, and (ii) generate weights that maximize classification error. This approach borrows principles from generative adversarial networks (GANs) and more generally from likelihood-free inference aiming to exploit the power of classifiers for discrepancy measure estimation. Embodiments may include a non-parametric method for generating weights, which compared to Inverse probability weighting (IPW), leads to a more uniform distribution of weights, and consequently to smaller confidence intervals.

For example, in an embodiment, a computer-implemented method for determining treatment effects may comprise receiving data, by computer program instructions executed by the processor, relating to treatment effects of at least one treatment on a plurality of persons in a plurality of treatment groups, wherein the data for each treatment group forms a dataset, the data generated by treating the plurality of persons with the at least one treatment and observing each person to determine effects of the at least one treatment on each person, reweighting, by computer program instructions executed by the processor, at least some of the datasets to balance biases in the data among the datasets by: determining a bias between at least two datasets using a classification error; and generating balancing weights for at least one of the datasets to reduce the bias between the two dataset, and determining, by computer program instructions executed by the processor, treatment effects using at least one reweighted dataset.

In embodiments, the bias between the at least two datasets is smaller when the classification error is larger. Balancing weights may be generated using an adversarial balancing framework. The adversarial balancing framework may comprise training a discriminator to minimize a classification error of the dataset; and generating weights to maximize the classification error of the dataset. The adversarial balancing framework may comprise iteratively: training a discriminator to minimize a classification error of the dataset; and generating weights to maximize the classification error of the dataset. The classification error is determined by the predictions of the discriminator.

In an embodiment, a system for determining treatment effects may comprise a processor, memory accessible by the processor, and computer program instructions stored in the memory and executable by the processor to perform: receiving data, by computer program instructions executed by the processor, relating to treatment effects of at least one treatment on a plurality of persons in a plurality of treatment groups, wherein the data for each treatment group forms a dataset, the data generated by treating the plurality of persons with the at least one treatment and observing each person to determine effects of the at least one treatment on each person, reweighting at least some of the datasets to balance biases in the data among the datasets by: determining a bias between at least two datasets using a classification error; and generating balancing weights for at least one of the datasets to reduce the bias between the at least two dataset, and determining treatment effects using at least one reweighted dataset.

In an embodiment, a computer program product for determining treatment effects may comprise a non-transitory computer readable storage having program instructions embodied therewith, the program instructions executable by a computer, to cause the computer to perform a method comprising: receiving data, by computer program instructions executed by the processor, relating to treatment effects of at least one treatment on a plurality of persons in a plurality of treatment groups, wherein the data for each treatment group forms a dataset, the data generated by treating the plurality of persons with the at least one treatment and observing each person to determine effects of the at least one treatment on each person, reweighting at least some of the datasets to balance biases in the data among the datasets by: determining a bias between at least two datasets using a classification error; and generating balancing weights for at least one of the datasets to reduce the in bias between the at least two dataset, and determining treatment effects using at least one reweighted dataset.

### BRIEF DESCRIPTION OF THE DRAWINGS

The details of the present invention, both as to its structure and operation, can best be understood by referring to the accompanying drawings, in which like reference numbers and designations refer to like elements.

FIG. 1 illustrates an exemplary system in which the embodiments of the present systems and methods may be implemented.

FIG. 2 is an exemplary flow diagram of a process, which may implement embodiments of the present methods, and which may be implemented in embodiments of the present systems.

FIG. 3 is an exemplary flow diagram of a process, which may implement an adversarial framework according to embodiments of the present methods, and which may be implemented in embodiments of the present systems.

FIG. 4 is an exemplary block diagram of a computer system in which processes involved in the embodiments described herein may be implemented.

### DETAILED DESCRIPTION

Embodiments of the present systems and methods may provide techniques for using classification error as a measure of the similarity between two datasets Embodiments may utilize bi-level optimization of minimizing classification error on one hand, and on the other hand generating weights that maximize classification error and hamper the ability to distinguish between the two datasets. In embodiments, a model may be trained to discriminate between the treatment groups and use its classification error as a measure of similarity between them. The key idea is that the larger the classification error is, the more indistinguishable treatment groups are. Embodiments may include a new framework for generating balancing weights that uses a bi-level optimization to alternately: (i) train a discriminator to minimize classification error, and (ii) generate weights that maximize classification error. This approach borrows principles from generative adversarial networks (GANs) and more generally from likelihood-free inference aiming to exploit the power of classifiers for discrepancy measure estimation. Embodiments may include a non-parametric method for generating weights, which compared to Inverse probability weighting (IPW), leads to a more uniform distribution of weights, and consequently to smaller confidence intervals.

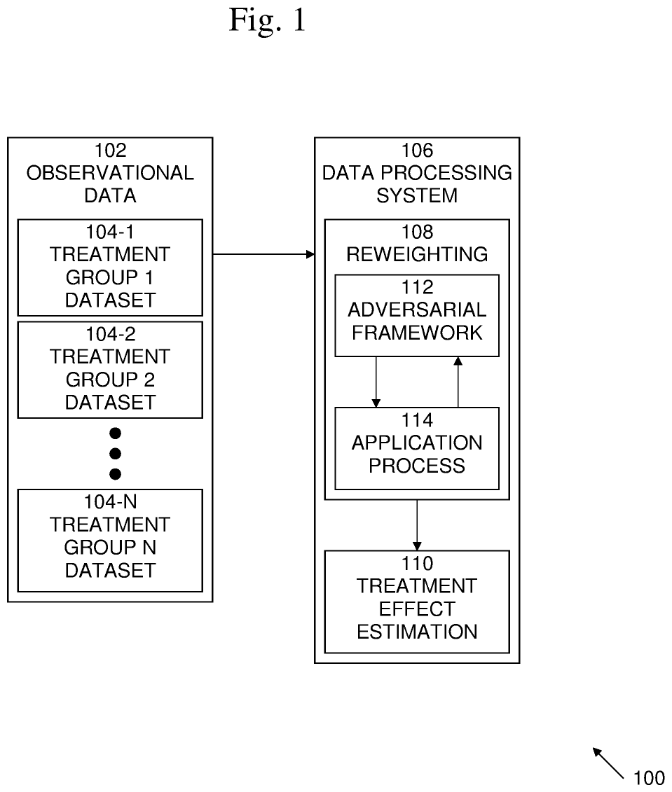

An exemplary system 100 in which embodiments of the present systems and methods may be implemented is shown in FIG. 1. In this example, observational data 102 may include observational data relating to a plurality of treatment groups, such as treatment group 1 dataset 104-1, treatment group 2 dataset 104-2, treatment group N dataset 104-N, etc. Observational data 102 may be processed by data processing system 106, which may include reweighting software 108, which may reweight datasets 104-1-N. In embodiments, classifiability may be used as a discrepancy measure for generating weights that balance the bias between datasets, such as datasets 104-1-N. Embodiments of the adversarial balancing framework may utilize the wealth and power of classification algorithms for comparing data distributions, which may be very complex and high dimensional. After reweighting, the datasets may be input to treatment effect estimation software 110.

For example, consider a population where each individual received a single treatment from a finite set of treatments A. The received treatment and the resulting outcome for every individual are indicated by the covariates A and Y, respectively. For every treatment a∈A, Y^{a} denotes the potential outcome for the treatment. This outcome is observed only when A=a, that is,

$$Y = {\sum }_{a} {Y}^{a} \cdot {1}_{A = a} .$$

Let X denote the vector of observed pre-treatment covariates used to characterize the individuals, and denote by p its distribution in the population. The expected outcome of a treatment a∈A in the population is:

$$E [ {Y}^{a} ] ≡ {\int }_{x} {\int }_{y} y \cdot p ( {Y}^{a} = y \vert X = x ) p ( X = x ) dydx ≡ {E}_{X \sim p ( X )} [ {E}_{{Y}^{a} \sim p ( {Y}^{a} \vert X )} [ {Y}^{a} \vert X ] ] . \qquad ( 1 )$$

The goal of many observational studies is to estimate E[Y^{a}] from a finite data sample D={(xᵢ,aᵢ,yᵢ)}ᵢ₌₁ⁿ. Conceptually, a straightforward estimation of E[Y^{a}] could be made by sampling Y^{a} from a population in which X˜p(X). However, Y^{a} is observed only in the subpopulation that actually received treatment a, and in which X˜p(X|A=a)≢p(X). To overcome this hurdle, the standard assumptions of strong ignorability: Y^{a}ΨA|X, and positivity: 0<p(A=a|X=x)<1, ∀a∈A may be employed. Strong ignorability, often stated as “no hidden confounders”, means that the observed covariates contain all the information that may affect treatment assignment. These assumptions allow rewriting Equation 1 as:

E[Y^{a}]=E_{X˜p(X)}[E_{Y˜p}(Y|X,A=a)[Y|X,A=a]]. (2)

Equation 2 suggests that E[Y^{a}] can be estimated by a sample from the subpopulation corresponding to A=a under the condition that the sample is distributed according to p(X) rather than the actual distribution p(X|A=a). A common approach to handle this sampling challenge is to assign a weight ω^{a}(x) to each individual characterized by x in the subpopulation under treatment a such that p(X=x|A=a)ω^{a}(x)=p(X=x). The weights that satisfy this condition are

$${ω}^{a} ( x ) = \frac{p ( A = a )}{p ( A = a \vert X = x )} ,$$

and therefore

E[Y^{a}]=E_{X˜p(X|A=a)}[E[Y|X=x,A=a]ω^{a}(x)]. (3)

Weights may be considered as balancing if they balance the biases between the distributions p(X|A=a) and p(X), making them more similar to each other. Given a finite sample D, the ultimate goal of all balancing methods is to produce weights wᵢ that approximate ω^{a}(xᵢ). IPW estimates wᵢ by learning a model for p(A=a|X=x) from D and using the inverse of the estimated probabilities, which may be unstable. However, embodiments of the present systems and methods may attempt to infer the weights directly. Following Equation 3, given wᵢ, the following estimation for the expected potential outcome may be used by embodiments:

$$E [ {Y}^{a} ] = {\sum }_{i : {A}_{i} = a} {w}_{i} {y}_{i} . \qquad ( 4 )$$

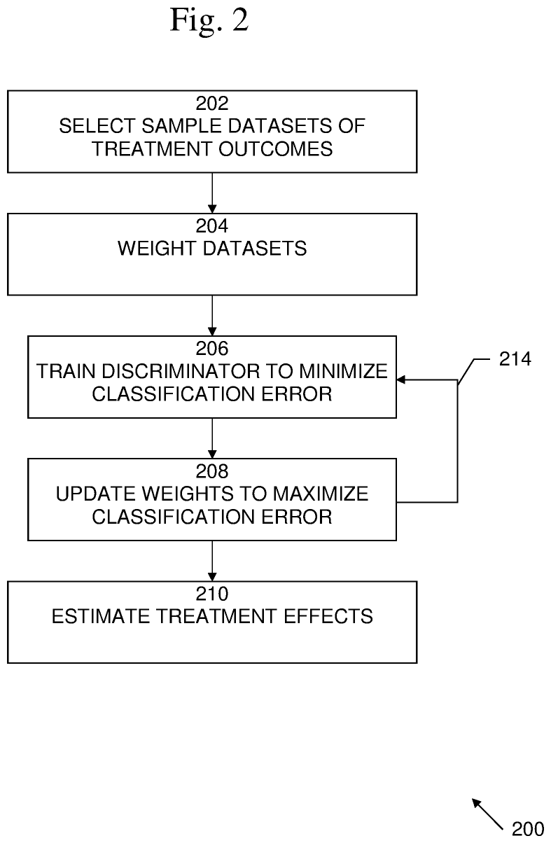

As shown in FIG. 1, embodiments of the present systems and methods may include an adversarial framework 112 for generating balancing weights, and a novel process 114 that applies the adversarial framework. An exemplary process 200 for estimating treatment effects, according to embodiments of the present methods, and which may be implemented by embodiments of the present systems, is shown in FIG. 2. It is best viewed in conjunction with FIG. 1. Process 200 begins with 202, in which one or more sample datasets 104-1-N of treatment outcomes may be selected. At 204, sample datasets 104-1-N may be weighted to balance the bias between datasets.

In embodiments, the goal is to generate a sample that resembles data coming from a true distribution p(X). However, while in the original GAN framework the generated sample is simulated by applying a transformation on unlimited random data, in embodiments, the balancing framework may be constrained to reweight a finite sample from p(x|a).

Given a finite sample from the distribution p(x|a), embodiments may find weights that maximize the probability of a discriminator to fail to distinguish the reweighted sample from an unweighted sample from p(X). Label the finite sample from the distribution p(x|a) as “1” and the sample from p(X) as 0. Let l(d (X), a) be a loss function measuring the classification error of d (X), the prediction of the discriminator, with respect to the label a, where a∈{0,1}. The adversarial objective may be adapted for this purpose as follows. Suppose [symbol] (x) is a non-negative function that reweights samples from p(x|a), resulting in a new distribution qₐ(x)≡ [symbol] (x)p(x|a). For qₐ(x) to be a valid density function, ω^{a}(x) should satisfy the constraint [symbol] _{X˜p (X|a)}[ω^{a}(x)]=1. Plugging qₐ into the generator distribution of the GAN framework and into the loss defined in this framework obtains

L(w^{a},d)= [symbol] _{X˜p(X)}[l(d(X),0)]+ [symbol] _{X˜q}_{^{a}}_{(X)}l(d(X,1)]

Since p(x|a) is a density function, we can rewrite the second term as follows:

L( [symbol] ,d)= [symbol] _{X˜p(X)}[l(d(X),0)]+ [symbol] _{X˜p(X|a)} [symbol] (X)·l(d(X,1)] (5)

The representation of [symbol] may be confined to a family of models and the loss may be optimized with respect to this family However, for the estimation problem defined in Equation 4, it suffices to infer point estimates of ω^{a} for the given sample from p(X|A=a). This approach may be taken without making any parametric assumptions on the functional form of ω^{a}. Denoting these estimates by wᵢ≡ [symbol] (xᵢ), the loss function becomes

$${L}_{n} ( w , d ; a ) = \frac{1}{n} {\sum }_{i = 1 , … , n} l ( d ( {x}_{i} ) , 0 ) + \frac{1}{{n}_{a}} {\sum }_{i : {A}_{i} = a} {w}_{i} l ( d ( {x}_{i} ) , 1 ) , \qquad ( 6 )$$

where w≡[wᵢ]_{i:A}_{ᵢ}₌ₐ are the weights corresponding to the individuals with A=a, and nₐ as their number. Note this expression is the empirical loss of a learning algorithm for the defined prediction task. Also, Equation 6 gives similar importance to classification errors in the sample from p(X) and the reweighted subsample corresponding to A=a. Since E_{p(x|a)}[ω^{a}(x)]=1 require that the generated weights satisfy the normalization property

$$\frac{1}{{n}_{a}} {\sum }_{i : {A}_{i} = a} {w}_{i} = 1.$$

The aim of the discriminator is to minimize the loss in Equation 6. Weights that lead to a failure of the discriminator to distinguish between the reweighted and unweighted samples will result in a maximization of the minimal loss. Therefore, we formulate the objective of the adversarial balancing framework as solving the following optimization problem:

$${w}^{*} = {arg max}_{w} ( \begin{matrix}min {L}_{n} ( w , d ; a ) \\ d\end{matrix} ) , such that w ∈ {Δ}^{{n}_{a}} , \qquad ( 7 )$$

where Δⁿ^{ₐ}={w|w[i]≥0, ∥w∥₁=nₐ} is the nₐ-dimensional simplex. This is a general framework for estimating the expected potential outcome for a given treatment; therefore, it can be used to estimate different types of causal effects. For example, the average treatment effect (ATE) is defined as E[Y¹]−E[Y⁰]. Another example is the average treatment effect in the treated (ATT), which is defined as E[Y¹|A=1]−E[Y⁰|A=1]. We estimate E[Y⁰|A=1] by reweighting the data sample from the untreated and using the data from treated individuals as the true data sample for comparison.

To search for a solution to the max-min objective in Equation 7, an iterative process may be used. At 206, a discriminator may be trained to minimize the empirical loss classification error as determined by Equation 6. At 208, the weights wᵢ may be updated to maximize the classification error using a single step of exponentiated gradient descent, which maintains the weight normalization constraint.

Embodiments may estimate E[Y^{a}] in the general population, for example, as follows. Let Dₐ be the data sample of the population under treatment a, that is, Dₐ={xᵢ:Aᵢ=a}. The data sample from the true distribution is Dₚₒₚ={xᵢ}ᵢ₌₁ⁿ. Note that by this definition Dₐ⊂Dₚₒₚ. However, our framework can be applied to different definitions of Dₐ and Dₚₒₚ. For example, to estimate E[Y⁰|A=1], define Dₐ{xᵢ:Aᵢ=0} and Dₚₒₚ={xᵢ:Aᵢ=1}. Given an initial set of weights {wᵢ} for the unit samples in Dₐ, define the augmented labeled dataset D by assigning a class label 1 and weights wᵢ to Dₐ, and a class label 0 to Dₚₒₚ:

D≡{(xⱼ,0;wⱼ=1)|xⱼ∈Dₚₒₚ}∪{(xᵢ,1;wᵢ)|xᵢ∈Dₐ}. (8)

Note that the unit samples in Dₚₒₚ are unweighted (wⱼ=1). The discriminator predicts the class label, z, of the samples in D using a classification algorithm d(x). Recall that the final objective of the adversarial framework is to find w that maximizes the objective in Equation 7. Following Equation 6, for a fixed classifier d, the generator's loss is linear in w and

$$\frac{\partial {L}_{n}}{\partial {w}_{i}} = l ( d ( {x}_{i} ) , 1 )$$

is constant. To maximize the objective in Equation 7, which refers to any classifier from the considered family, update the weights using a single step of exponentiated gradient ascent:

$${w}_{i}^{t + 1} = {n}_{a} \frac{{w}_{i}^{t} exp ( α \cdot l ( d ( {x}_{i} ) , 1 ) )}{{\sum }_{j} {w}_{j}^{t} exp ( α \cdot l ( d ( {x}_{j} ) , 1 ) )} \qquad ( 9 )$$

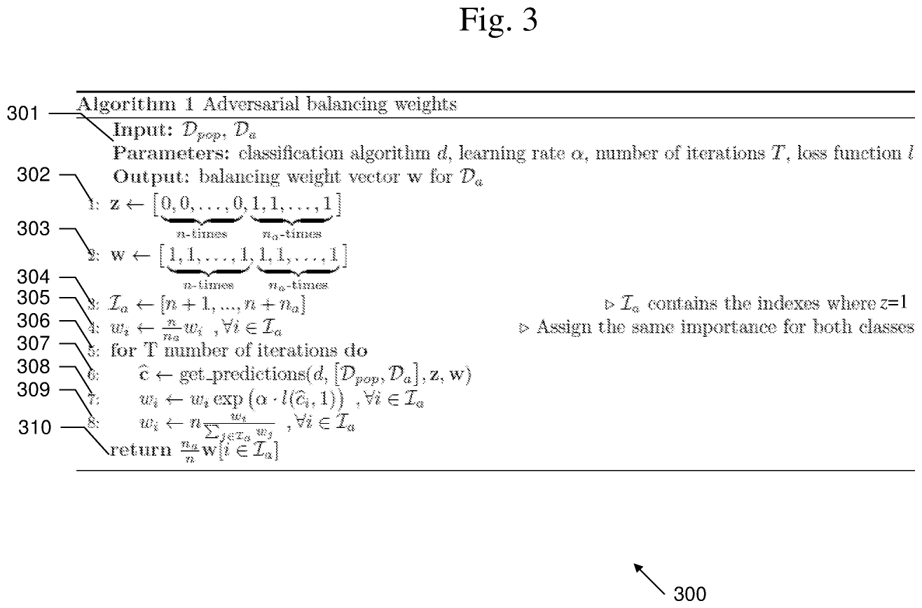

A pseudocode example of a process 300 for the adversarial framework for non-parametric generation of balancing weights is shown in FIG. 3. Process 300 begins with 301, in which input dataset Dₚₒₚ and Dₐ may be input to process 300. Parameters that may control the processing performed by process 300 may include a classification algorithm d, a learning rate α, a number of iterations T, and a loss function l. The output of process 300 may include a vector of balancing weights w for Dₐ. At 302, a vector z may be defined including n zeroes followed by nₐ ones. At 303, an initial vector w may be defined including n plus nₐ ones. At 304, Iₐ may be defined, wherein Iₐ may include the indexes of the values in z that include ones, that is Iₐ←[n+1, . . . , n+nₐ]. At 305, wᵢ may be assigned with

$$\frac{n}{{n}_{a}} {w}_{i} , ∀ i ∈ {I}_{a} ,$$

At 306, the process may iterate 307-309 for a number of iterations T. At 307, ĉ may be assigned with the results of get_predictions(d, [Dₚₒₚ, Dₐ], z, w). The function get-predictions may use the classification algorithm d to determine the current classification error of the datasets Dₚₒₚ, and Dₐ using the vectors z, and w. At 308, wᵢ may be assigned with wᵢ exp(∝·l(ĉᵢ, 1)), ∀i∈Iₐ. At 309, wᵢ may be assigned with

$$n \frac{{w}_{i}}{{\sum }_{j ∈ {I}_{a}} {w}_{j}} , ∀ i ∈ {I}_{a} ,$$

After T iterations, at 310, process 300 may return

$$\frac{n}{{n}_{a}} w [ i ∈ {I}_{a} ] ,$$

which may be a balancing weight vector w for Dₐ.

In embodiments, only the weights for Dₐ may be updated, while weights for the sample units in Dₚₒₚ may be constantly set to 1. In each iteration the sum of weights in Dₐ equals n, ensuring the same importance with respect to the discriminator loss. The predictions of the discriminator at 307 maybe be obtained with cross validation, to better approximate the generalization error in Equation 5.

The choice of the classifiers family and its hyper-parameters is important to enable approximation of the minimal loss defined in Equation 5 with the empirical loss in Equation 6. In embodiments, the family of classifiers may be rich enough to distinguish between “non-similar” (weighted) datasets. The classification error is determined by the predictions of the discriminator. using a classifier that is not over-fitted or under-fitted.

Returning to FIG. 2, at 210, treatment effect may be estimated using the reweighted sample datasets.

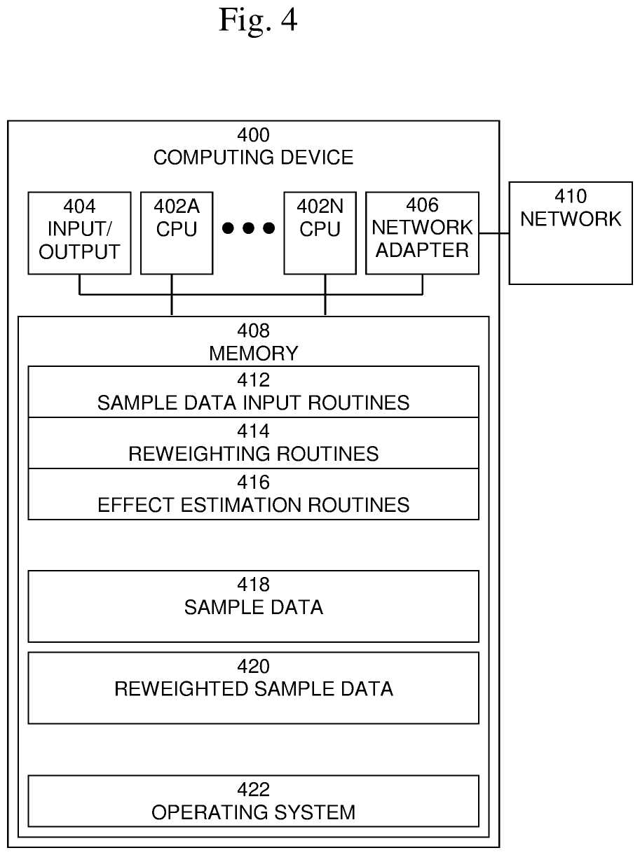

An exemplary block diagram of a computer system 402, in which processes involved in the embodiments described herein may be implemented, is shown in FIG. 4. Computer system 402 may be implemented using one or more programmed general-purpose computer systems, such as embedded processors, systems on a chip, personal computers, workstations, server systems, and minicomputers or mainframe computers, or in distributed, networked computing environments. Computer system 402 may include one or more processors (CPUs) 402A-402N, input/output circuitry 404, network adapter 406, and memory 408. CPUs 402A-402N execute program instructions in order to carry out the functions of the present communications systems and methods. Typically, CPUs 402A-402N are one or more microprocessors, such as an INTEL CORE® processor. FIG. 4 illustrates an embodiment in which computer system 402 is implemented as a single multi-processor computer system, in which multiple processors 402A-402N share system resources, such as memory 408, input/output circuitry 404, and network adapter 406. However, the present communications systems and methods also include embodiments in which computer system 402 is implemented as a plurality of networked computer systems, which may be single-processor computer systems, multi-processor computer systems, or a mix thereof.

Input/output circuitry 404 provides the capability to input data to, or output data from, computer system 402. For example, input/output circuitry may include input devices, such as keyboards, mice, touchpads, trackballs, scanners, analog to digital converters, etc., output devices, such as video adapters, monitors, printers, etc., and input/output devices, such as, modems, etc. Network adapter 406 interfaces device 400 with a network 410. Network 410 may be any public or proprietary LAN or WAN, including, but not limited to the Internet.

Memory 408 stores program instructions that are executed by, and data that are used and processed by, CPU 402 to perform the functions of computer system 402. Memory 408 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 408 may vary depending upon the function that computer system 402 is programmed to perform. In the example shown in FIG. 4, exemplary memory contents are shown representing routines and data for embodiments of the processes described above. However, one of skill in the art would recognize that these routines, along with the memory contents related to those routines, may not be included on one system or device, but rather may be distributed among a plurality of systems or devices, based on well-known engineering considerations. The present communications systems and methods may include any and all such arrangements.

In the example shown in FIG. 4, memory 408 may include sample data input routines 412, reweighting routines 414, effect estimation routines 416, sample data 418, reweighted sample data 420, and operating system 422. Sample data input routines 412 may include software routines to obtain and receive sample data such as observational data relating to treatment outcomes including datasets of different treatment groups. Reweighting routines 414 may include software routines to reweight datasets, as described above. Effect estimation routines 416 may include software routines to estimate effects of treatments based on reweighted datasets of observed treatment outcomes, as described above. Sample data 418 may include original datasets of observed treatment outcomes. Reweighted sample data 420 may include reweighted datasets of observed treatment outcomes. Operating system 422 may provide overall system functionality.

As shown in FIG. 4, the present communications systems and methods may include implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

What is claimed is:

1. A method for determining treatment effects implemented in a computer comprising a processor, memory accessible by the processor, and computer program instructions stored in the memory and executable by the processor, the method comprising:
receiving data, by computer program instructions executed by the processor, relating to treatment effects of at least one treatment on a plurality of persons in a plurality of treatment groups, wherein the data for each treatment group forms a dataset, the data generated by treating the plurality of persons with the at least one treatment and observing each person to determine effects of the at least one treatment on each person;
reweighting, by computer program instructions executed by the processor, at least some of the datasets to balance biases in the data among the datasets by:
determining a bias between at least two datasets using a classification error;
and
generating balancing weights for at least one of the datasets to reduce the bias between the two dataset; and
determining, by computer program instructions executed by the processor, treatment effects using at least one reweighted dataset.

2. The method of claim 1, wherein the bias between the at least two datasets is smaller when the classification error is larger.

3. The method of claim 1, wherein balancing weights are generated using an adversarial balancing framework.

4. The method of claim 3, wherein the adversarial balancing framework comprises:
training, by computer program instructions executed by the processor, a discriminator to minimize a classification error of the dataset; and
generating, by computer program instructions executed by the processor, weights to maximize the classification error of the dataset.

5. The method of claim 3, wherein the adversarial balancing framework comprises iteratively:
training, by computer program instructions executed by the processor, a discriminator to minimize a classification error of the dataset; and
generating, by computer program instructions executed by the processor, weights to maximize the classification error of the dataset.

6. The method of claim 5, wherein:
the classification error is determined by the predictions of the discriminator. using a classifier that is not over-fitted or under-fitted.

7. A system for determining treatment effects, the system comprising a processor, memory accessible by the processor, and computer program instructions stored in the memory and executable by the processor to perform:
receiving data, by computer program instructions executed by the processor, relating to treatment effects of at least one treatment on a plurality of persons in a plurality of treatment groups, wherein the data for each treatment group forms a dataset, the data generated by treating the plurality of persons with the at least one treatment and observing each person to determine effects of the at least one treatment on each person;
reweighting at least some of the datasets to balance biases in the data among the datasets by:
determining a bias between at least two datasets using a classification error;
and
generating balancing weights for at least one of the datasets to reduce the bias between the at least two dataset; and
determining treatment effects using at least one reweighted dataset.

8. The system of claim 7, wherein the bias between the at least two datasets is smaller when the classification error is larger.

9. The system of claim 7, wherein balancing weights are generated using an adversarial balancing framework.

10. The system of claim 9, wherein the adversarial balancing framework comprises:
training a discriminator to minimize a classification error of the dataset; and
generating weights to maximize the classification error of the dataset.

11. The system of claim 9, wherein the adversarial balancing framework comprises iteratively:
training a discriminator to minimize a classification error of the dataset; and
generating weights to maximize the classification error of the dataset.

12. The system of claim 11, wherein:
the classification error is determined by the predictions of the discriminator.

13. A computer program product for determining treatment effects, the computer program product comprising a non-transitory computer readable storage having program instructions embodied therewith, the program instructions executable by a computer, to cause the computer to perform a method comprising:
receiving data, by computer program instructions executed by the processor, relating to treatment effects of at least one treatment on a plurality of persons in a plurality of treatment groups, wherein the data for each treatment group forms a dataset, the data generated by treating the plurality of persons with the at least one treatment and observing each person to determine effects of the at least one treatment on each person;
reweighting at least some of the datasets to balance biases in the data among the datasets by:
determining bias between at least two datasets using a classification error;
and
generating balancing weights for at least one of the datasets to reduce the bias between the at least two dataset; and
determining treatment effects using at least one reweighted dataset.

14. The computer program product of claim 13, wherein the bias between the at least two datasets is smaller when the classification error is larger.

15. The computer program product of claim 13, wherein balancing weights are generated using an adversarial balancing framework.

16. The computer program product of claim 15, wherein the adversarial balancing framework comprises:
training a discriminator to minimize a classification error of the dataset; and
generating weights to maximize the classification error of the dataset.

17. The computer program product of claim 15, wherein the adversarial balancing framework comprises iteratively:
training a discriminator to minimize a classification error of the dataset; and
generating weights to maximize the classification error of the dataset.

18. The computer program product of claim 17, wherein:
the classification error is determined by the predictions of the discriminator.
